(19) United States
(12) Patent Application Publication
(10) Pub. No.: US 2009/0210959 A1
(43) Pub. Date: Aug. 20, 2009

(54) METHOD FOR PRODUCTION OF SAPONARIA FROM MICROSPORES

(75) Inventors: Alison M.R. Ferrie, Saskatoon (CA); John Balsevich, Saskatoon (CA); Zoe Ehlert, Saskatoon (CA)

Correspondence Address: SUGHRUE MION, PLLC, 2100 PENNSYLVANIA AVENUE, N.W., SUITE 800, WASHINGTON, DC, 20037, US

(73) Assignee: NATIONAL RESEARCH COUNCIL OF CANADA, Ottawa (CA)

(21) Appl. No.: 11/913,418

(22) PCT Filed: May 4, 2006
(86) PCT No.: PCT/CA2006/000725

§ 371 Date: Jun. 20, 2008

Related U.S. Application Data
(60) Provisional application No. 60/677,952, filed on May 4, 2005.

(51) Int. Cl. A01H 4/00; A01H 5/00; A01H 1/02

(52) U.S. Cl. 800/263; 800/268; 800/298

(57) ABSTRACT

Saponaria vaccaria, related Saponaria species and species in other genera of the Caryophyllaceae family are well known for the production of saponins which have many established commercial uses including: detergents, foaming agents, emulsifiers and recently as adjuvants for vaccine development. Additionally, seed of Saponaria species comprises a novel starch with uniform small size grains suitable for numerous novel applications, and various cyclopeptides, proteins and secondary metabolites that have potential drug, nutraceutical or health care properties. The present invention discloses a method of culturing isolated microspores and generation of doubled haploid plant lines that are suitable for the rapid selection of Saponaria species with altered and improved seed composition and agronomic performance.

### PRIOR APPLICATION INFORMATION

This application claims the benefit of U.S. Provisional Application 60/677,952, filed May 4, 2005.

### TECHNICAL FIELD

The invention relates generally to biotechnology. More particularly, the present invention relates to methods for the generation of doubled haploid plants from microspores and novel compositions of matter so derived.

### BACKGROUND

Saponaria vaccaria (=Vaccaria hispanica, V. segetalis, V. pyramidata) is a herbaceous dicotyledonous annual in the family Caryophyllaceae, commonly known as cow cockle. This and related species have been known since antiquity as Soapworts as the leaves and other parts of these plants contain saponins—natural detergents and foaming agents.

Saponins are a structurally diverse class of glycosides comprising a central aglycon, (non-sugar portion) which is typically a triterpenoid with sugars attached at one or two locations (C-3 and C-28) comprising the glycon. In addition to terpenoids, saponins may comprise a steroidal or steroidal alkaloid aglycon which may account for the pharmacological properties of plant extracts. The number of monosaccharide units attached, comprising linear or branched chains, varies from one to twelve sugars. Saponins with sugars attached at a single position are termed monodesmosides and at two positions bisdesmosides.

Saponins are found widely in many plant genera and include common food, flavor, nutraceutical or feed crops such as soybean, licorice, fenugreek, ginseng, quinoa, yucca and alfalfa where they are believed to provide protection to the plant from fungal or other pathogenic organisms.

The detergent nature of saponins and their ability to form stable foams and emulsions has led to the use of saponins in many commercial products that include: mild detergents for cleaning delicate fabrics and for easily damaged surfaces of oil paintings (Kulperger, U.S. Pat. No. 5,503,766), fire extinguishing foams (Meyer, U.S. Pat. No. 6,051,154), photographic emulsions, foaming agents for beer and soft drinks, agents to suppress acid mist during electrolytic recovery of heavy metals from ores (D'Arrigo, U.S. Pat. No. 4,684,479), and as components of animal feed. Saponins bind ammonia and help to suppress the stench associate with pig and chicken manure. The anti-microbial activity of saponins provides a positive health benefit in animal feeds. The saponins from the seed hulls of quinoa have been shown to protect seeds and plants from bacterial (Dutcheshen, U.S. Pat. No. 6,743,752) and fungal (Dutcheshen et al., U.S. Pat. No. 6,482,770) pathogens.

In addition to these established uses of saponins, recent attention has focused on the potential of saponins as drugs, nutraceutical healthcare products, and vaccine adjuvants and drug delivery products (liposomes). Investigation of the potential of saponins as adjuvants has been a significant recent activity. Much of the research on adjuvants has focused on saponins from the soapbark tree (Quillaja saponaria) indigenous to South America.

Additional sources of saponins for adjuvant development include Polygala senega, Chenopodium quinoa, and Saponaria sp.

In addition to use as adjuvants, saponins have been considered for drug delivery uses as components of delivery vehicles such as liposomes.

In addition to the physical properties of saponins for the formation of foams and emulsions, saponins have many potential direct health care applications, well-known drugs such as the heart drug digitoxin (foxglove) and estrogenic steroids (true yam) are saponins. Other health care applications of saponins include: regulation of apoptosis, blood related illnesses, treatment of premenstral syndrome, neurological diseases, dementia, Alzheimer's disease, cardiovascular disease, cancer, diabetes, regulation of cholesterol, regulation of inflammation, and treatment of alcoholism.

Saponin containing materials have well established nutraceutical and topical skin care applications that include: renewal of the epidermis and stimulation of hair growth, treatment of microbial infections, treatment of viral infections, removal of head lice, use in organic toothpaste, and as a base for the production of steroidal-drugs.

In addition to saponins, the seed of Saponaria vaccaria contains numerous other fractions of commercial interest. The majority of the seed (60-65%) includes an extremely fine starch with particle size in the 0.5-1.5μ range (Biladeris, C. G., et al., Starch/Stärke 45:121-127, 1993). The extremely small size and uniformity of particle shape provides this starch with unique properties that can be harnessed and adapted to novel applications.

Starches, modified starches, and starch-based compositions have been used extensively to develop low fat or reduced calorie foods. Saponaria and other fine starches add desirable textural qualities to foodstuffs and generate a creamy feel and texture resulting from the innately small particle size that generates the slippery effect. This property was recognized by the food industry and Saponaria and other small-size starches have been used in specialty creams and puddings as a fat substitute (O'Rourke et al., U.S. Pat. No. 4,438,148; Camin et al., U.S. Pat. No. 6,485,775; Singer, U.S. Pat. No. 5,370,894; Singer et al., U.S. Pat. Nos. 4,911,946 and 5,153,020; Mazza, G., et al., J. Agric. Food Chem., 40:1520-1523, 1992).

The small particle size starch textural and hydration properties have also found use in cosmetic applications largely in body powders and deodorants (i.e., Matesevac, et al., U.S. Pat. No. 6,267,970; McCuaig, U.S. Pat. No. 5,662,937; Whistler, U.S. Pat. No. 5,453,281).

Saponaria seed also contains 12-14% protein with an amino acid balance that is nutritional and suitable for human and animal food. (Mazza, G., et al., J. Agric. Food Chem. 40:1520-1523, 1992.) In addition to seed storage proteins, Saponaria additionally produces a range of cyclopeptides that may comprise several percent of the seed weight. Cyclopeptides are not commonly produced by plants and such peptides are highly stable and frequently have interesting biochemical properties. Cyclic peptides, both naturally occurring and synthetically derived are well known for antifungal, antibacterial, and antiviral properties. Additional medical and therapeutic applications include anti-cancer, anti-inflammatory, anti-thrombosis, and anti-diuretic activity.

Saponaria seed products are sold in China as a nutraceutical composition for treatment of “women's problems” and as a tonic for lactating mothers. Cyclopeptides identified as segetalin A, cyclo (gly-val-pro-val-trp-ala) and segetalin B, cyclo (gly-val-ala-trp-ala) exhibit estrogenic activity in rats (Morita et al., 1994, 1995). Additionally segetalin E, cyclo (gly-tyr-val-pro-leu-trp-pro) exhibits moderate leukemia cell division activity. Other substances of potential nutraceutical or pharmacological interest from Saponaria seed include phenolics and xanthones with anti-oxidant properties.

Saponaria vaccaria has many attributes that could make this species of interest as a new starch and protein crop and as a source of beneficial healthcare and nutraceutical products. It is also noteworthy that no Caryophyllaceae species have been developed as major food or feed crops. Saponaria has been used for minor uses in specialized foods, whereas additional members of the family, such as carnation and Babysbreath, have been bred only as ornamentals.

### SUMMARY OF THE INVENTION

The present invention discloses methods and procedures for the rapid production of numerous microspore derived lines of Saponaria vaccaria that can be used for development and breeding of new plant lines with improved agronomic performance and composition. In particular, variants of saponin, starch, and secondary metabolite composition are of interest and constitute a preferred embodiment of the invention.

The use of doubled-haploid plants as a vehicle for plant breeding is well established and has become a routine practice for crops such as canola, wheat, barley, and maize. The main advantage of cell culture techniques for generating doubled-haploid plants is the greatly reduced time required to achieve homozygosity; years of selfing and recurrent selection are replaced by a single culture cycle. The use of haploid technologies results in fixing of traits, which allows for efficient screening and selection of desirable phenotypes.

Haploid plants that comprise only a single set of chromosomes are infertile and must be doubled in chromosome complement before use in breeding. Techniques for doubling the chromosome number of haploid plants using colchicine and other chemicals that disturb the cytoskeleton of cells are well known.

Haploids can be produced by a number of methods.

Haploid plants occur with low frequency naturally and can be identified in field grown populations based on examination of flower morphology. The low frequency of occurrence makes this approach impractical (Bosemark et al., U.S. Pat. No. 5,639,951).

Haploid plants may result from wide hybridization followed by chromosome elimination. This is the basis of the Hordeum bulbosum technique where common barley, Hordeum vulgare is crossed with H. bulbosum with the subsequent elimination of the H. bulbosum chromosomes. This method has been used to develop barley, wheat, maize, sorghum, and millet cultivars but has no use outside of cereals.

Gynogenesis involves the culture of female cells, unfertilized ovaries or ovules. This method has only been shown to work with a few species and the frequency of embryo formation is low (Dirks, U.S. Pat. No. 5,492,827).

Androgenesis involves the culture of developing microspores, which includes both culture of the entire anther housing the developing microspores or the physical disruption of anthers and the culture of the isolated microspore cells.

The development of embryos, haploid and doubled-haploid plants from developing microspores in culture has been achieved to date in various species (Dunwell, 1986; Ferrie et al., 1994) however, many more remain to be investigated. Techniques to generate large numbers of embryos from cultured microspores have only been reported for a few species. It is now well known in the literature that a large variety of factors influence the success of inducing embryo development from isolated microspores or from anther cultures (Ferrie, A. M. R., et al., In Vitro Embryogenesis in Plants, 309-344, 1995; Maheshwari, S. C., et al., Amer. J. Bot. 69:865-879, 1982).

It appears that one important aspect of methods for inducing embryo development from microspores is to disrupt the developmental process using physical or chemical means. The disruption must coincide with the developmental stage of the microspore that subsequently allows embryo formation. Typically, the stage that is disrupted is during the late uninucleate to early bi-nucleate stage of development (Gaillard, A., et al., Plant Cell Reports 10:55-58, 1991; Kott, L. S., et al., Can. J. Bot. 66:1658-1664, 1988; Fan, Z. et al., Protoplasma 147:191-199, 1988). The chief agent for disruption has been elevated temperatures (Keller and Armstrong, 1978; Cordewener et al., 1994) but chemicals such as colchicine, cytochalasin B, and trifluralin that are known to disturb cellular cytoskeleton organization have also been shown to be effective (Simmonds et al., U.S. Pat. Nos. 5,900,375 and 6,200,808).

Another aspect that has been shown to be important for recovery of embryos from induced microspores is the nutrient medium. Both the composition of the medium and the amount of carbohydrates have been shown to be critical factors for some applications. High concentrations of sucrose (13%) or other specific sugars such as maltose have been shown to be important, however species differ greatly in the composition of the medium that is optimal for embryo induction. In addition to sugars and salts, plant growth regulators such as auxins, cytokinins, or gibberellins may be required. Various gametocidal chemicals such as 2-hydroxynicotinic acid, 2-chloroethyl-phosphonic acid and pronamide and undefined natural factors emanating from ovules (Konzak et al., U.S. Pat. Nos. 6,764,854 and 6,362,393) may also be required.

U.S. Pat. No. 4,840,906 to Hunter describes anther culture of barley microspores on media with varying sugar composition showing a stimulative effect of maltose. Spikes containing anthers were pretreated at 4° C. for a period of up to 28 days prior to culture.

U.S. Pat. Nos. 5,322,789 and 5,445,961 to Genovesi et al, teach isolated microspore and anther culture of corn. The method involves pre-treatment of microspores at 10° C., the requirement for the sugar alcohol mannitol and the chromosome doubling agent coichicine in the culture medium, transfer of cells through a series of media, the use of callus and support matrices for feeding developing embryos, and the use of auxins and cytokinins for regeneration of plantlets. This and other methods developed for cereals have the limitation that the methods may result in formation of significant numbers of non-green albino plants.

U.S. Pat. No. 6,362,393 to Konzak et al. describes a method for the production of doubled-haploid plants from wheat. The method involves subjecting developing microspores to temperature and nutrient stress, a medium comprised of mannitol, maltose, auxins, cytokinins and/or gibberellin plant growth regulators and a specific sporophytic development inducing chemical belonging to the class of chemicals used in whole plants to impart male sterility. Further, U.S. Pat. No. 6,764,854 to Konzak et al. describes an application of the above method for the production of doubled-haploid rice.

U.S. Pat. No. 6,812,028 to Kasha et al. teach a method for regeneration of isolated barley microspores that also includes low temperature pre-treatment but includes additional medium components comprising an arabinogalactan protein, auxins or unknown natural factors from ovaries.

Isolated microspore culture protocols have also been described for various Brassica species (i.e., B. napus, B. rapa, B. oleracea) by Ferrie A. M. R., et al., Plant Cell Reports 14:580-584, 1999; Ferrie A. M. R., et al., In Vitro Embryogenesis in Plants, 309-344, 1995; Ferrie A. M. R., et al., Biotechnology in Agriculture and Forestry, Vol. 54:149-168, 2004; Barro, F. et al., Plant Breeding 118:79-81, 1999 (B. carinata); Lionneton, E., et al., Plant Cell Reports 20:126-130, 2001 (B. juncea). Factors that have been identified that contribute to induction and development of microspore-derived embryos include growth conditions of the parent plants, stage of microspore development, temperature and carbohydrate composition of the medium. The requirement for temperature stress may be replaced by chemical inhibitors of cytoskeleton integrity (Simmonds et al., U.S. Pat. Nos. 5,900,375 and 6,200,808).

Despite the considerable body of literature describing successful development of embryos from microspores of numerous species, the methods as described have not worked at all in many instances. For instance, the very well studied model plant Arabidopsis thaliana is an example of a recalcitrant species that does not respond to methods that are known to work well with other species. Additionally it is well known by those experienced in the art, that response to microspore culture varies greatly from cultivar to cultivar and from plant to plant of the same cultivar suggesting unknown genetic influences.

In one embodiment, a method for producing doubled-haploid plants of Saponaria vaccaria comprises cultivating of microspore donor plants under conditions that allow the development of microspores capable of development into haploid embryos; isolating of microspores at the uninucleate—binucleate stage of development that can be induced to develop into embryos in culture; culturing isolated microspore cells in media with appropriate composition under culture conditions that induce embryo development in microspores; and generating doubled-haploid plantlets from microspore-derived embryos.

In another embodiment, a method for producing doubled-haploid plants of Saponaria vaccaria consists essentially of cultivating of microspore donor plants under conditions that allow the development of microspores capable of development into haploid embryos; isolating of microspores at the uninucleate—binucleate stage of development that can be induced to develop into embryos in culture; culturing isolated microspore cells in media with appropriate composition under culture conditions that induce embryo development in microspores; and generating doubled-haploid plantlets from microspore-derived embryos.

In an additional embodiment, a method for producing doubled-haploid plants of Saponaria vaccaria consists of cultivating of microspore donor plants under conditions that allow the development of microspores capable of development into haploid embryos; isolating of microspores at the uninucleate—binucleate stage of development that can be induced to develop into embryos in culture; culturing isolated microspore cells in media with appropriate composition under culture conditions that induce embryo development in microspores; and generating doubled-haploid plantlets from microspore-derived embryos.

According to a first aspect of the invention, there is provided a method of producing a microspore-derived doubled haploid plant of Saponaria vaccaria comprising:

growing microspore donor plants under conditions that allow the development of microspores competent for development into embryos;

isolating the microspores from flower buds comprising the microspores at a developmental stage competent for inducing embryo development;

culturing the isolated microspores in a medium with a composition that allows for the development of microspore-derived embryos;

exposing the cultured isolated microspores to an elevated temperature for inducing embryogenesis;

recovering embryos; and

generating doubled haploid plants.

According to a second aspect of the invention, there is provided a method of generating new varieties of Saponaria vaccaria comprising:

providing a first doubled haploid Saponaria vaccaria variety having a first desirable characteristic, said variety having been produced by the method described herein;

mating said first variety with a second Saponaria vaccaria variety; and

selecting at least one progeny having said first desirable characteristic.

According to a third aspect of the invention, there is provided a doubled haploid Saponaria vaccaria plant made according to the method as described herein.

### BRIEF DESCRIPTION OF THE DRAWINGS AND TABLES

FIG. 1: Effect of bud size on microspore embryogenesis of Saponaria vaccaria.

FIG. 2a: Effect of donor plant growth temperature on microspore embryogenesis of Saponaria vaccaria cv. White Beauty.

FIG. 2b: Effect of donor plant growth temperature on microspore embryogenesis of Saponaria vaccaria cv. Pink Beauty.

FIG. 3a: Effect of microspore density on microspore embryogenesis of Saponaria vaccaria cv. White Beauty.

FIG. 3b: Effect of microspore density on microspore embryogenesis of Saponaria vaccaria cv. Pink Beauty.

FIG. 4: Genotypic effect of microspore embryogenesis in Saponaria vaccaria.

FIG. 5: Different saponin “chemotypes” from two double haploid lines (Z-39 and Z-69).

FIG. 6: Hplc Chromatogram (non-polar cyclopeptide region) of seed extracts from two double haploid lines (Z-48 and Z-56).

Table 1: Effect of basal media on microspore embryogenesis in Saponaria vaccaria cv. White Beauty.

Table 2: Effect of different carbohydrates and concentration on microspore embryogenesis of Saponaria vaccaria cv. White Beauty.

Table 3a: Effect of culture conditions on microspore embryogenesis of Saponaria vaccaria cv. White Beauty.

Table 3b: Effect of culture conditions on microspore embryogenesis of Saponaria vaccaria cv. Pink Beauty.

Table 4: Relative amounts of major saponin components in seed extracts of double haploid lines Z-39 and Z-69.

Table 5: Relative amounts of major tryptophan containing cyclopeptides, segetalins A and B, in seed extracts of double haploid Lines Z48 and Z-56.

### DESCRIPTION OF THE PREFERRED EMBODIMENTS

Unless defined otherwise, all technical and scientific terms used herein have the same meaning as commonly understood by one of ordinary skill in the art to which the invention belongs. Although any methods and materials similar or equivalent to those described herein can be used in the practice or testing of the present invention, the preferred methods and materials are now described. All publications mentioned hereunder are incorporated herein by reference.

Isolated microspore culture represents a plant breeding tool that can be used to rapidly produce uniform homozygous lines that may be evaluated for improved agronomic performance and novel compositions. The present invention has utility as a tool for the development of improved variety development in a group of plant species with nutraceutical properties.

According to the invention, there is provided a method of producing a microspore-derived doubled haploid plant of Saponaria vaccaria comprising:

growing microspore donor plants under conditions that allow the development of microspores competent for development into embryos;

isolating the microspores from flower buds comprising the microspores at a developmental stage competent for inducing embryo development;

culturing the isolated microspores in a medium with a composition that allows for the development of microspore-derived embryos;

exposing the cultured isolated microspores to elevated temperatures for inducing embryogenesis;

recovering embryos; and

generating doubled haploid plants.

In another embodiment of the invention, there is provided a method of generating new varieties of Saponaria vaccaria comprising:

providing a first doubled haploid Saponaria vaccaria variety having a first desirable characteristic, said variety having been produced by the above-described method;

mating said first variety with a second Saponaria vaccaria variety; and

selecting at least one progeny having said first desirable characteristic.

In some embodiments, the second Saponaria vaccaria variety may be a doubled haploid generated according to the above-described method as well. In these embodiments, the second variety may have a second desirable characteristic and progeny may be selected which have both the first and second desirable characteristics. As will be appreciated by one of skill in the art, because doubled haploid plant(s) are being used, much less screening of progeny is required to isolate or select a progeny having the desired characteristic(s) compared to the use of natural varieties. As discussed below, these methods can be used to develop elite plants.

As will be appreciated by one of skill in the art, as used herein, ‘desired characteristic’ may refer to one trait or may refer to a plurality of traits, for example, a specific profile. It is of note that desirable traits will be readily apparent to one of skill in the art. Exemplary desirable traits may include but are by no means limited to increased or decreased starch content, vaccaroside B content, vaccaroside E content, segetoside I content, segetoside I Ac content, segetoside H content, segetalin B content, segetalin A content, saponin content and the like as well as combinations thereof, as discussed herein.

As will be appreciated by one of skill in the art, the Saponaria vaccaria may be any accession or variety known in the art, for example, but by no means necessarily limited to White Beauty, Pink Beauty, PI 304488, PI 578121 and PI 597,629.

The immature microspores will development into a pollen grain under normal, natural conditions. However, if the microspores are cultures and are given a stress treatment, they may develop into embryos. A general write-up on haploidy development may be found in Ferrie, A. M. R., Palmer, C. E., and Keller, W. A. 1995. Haploid embryogenesis. In: In vitro embryogenesis in plants. Thorpe, T. A. (ed.) Kluwer Academic Publishers, Dordrecht. p. 309-344, which is incorporated herein by reference for the description of haploidy development. In most cases, microspores are competent between the mid-late uninucleate—the early binucleate stage of development.

Although anther and isolated microspore culture has been reported to be successful for a variety of species, a large number of different factors have been identified that may influence success. In order to efficiently develop an effective procedure for a new species such as Saponaria vaccaria, a member of the previously untried Caryophyllaceae, the following strategy was employed.

It is well established that successful microspore culture methods require microspores to be at a developmental stage where they are competent to respond to embryo induction. This stage is typically the mid to late uninucleate stage of development just prior to the first microspore mitosis. The importance of the mitotic process is further confirmed by induction of embryo formation in some circumstances by chemicals such as colchicine that are known to inhibit microtubule formation of the spindle apparatus.

In many plant species the size of the flower bud may be used as a marker of microspore development as the majority of microspores develop at similar rates within the anther. For instance, in the well-studied species B. napus, the bud size correlated with the uninucleate stage of microspore development is 3-4 mm. It was observed in Saponaria that flower buds of less than 4 mm and greater than 8 mm in size were not as responsive as the other sizes, as discussed below. The bud size of Saponoria with the most responsive microspores was determined to be 4-7.9 mm. Specifically, at 7-7.9 mm, the result is 61% of the 6-6.9 mm buds, whereas, from 8-8.9, the response is 17% of the 6-6.9 mm buds. Accordingly, in some embodiments, donor plants are grown until the microspores are competent for the development into embryos, for example, until the bud size is between 3-9 mm, or between 3-8 mm or between 4-9 mm or between 4-8 mm or preferably between 4-7.9 mm or 4-6.9 mm.

It is expected that the conditions for the growth of donor plants and pre-conditioning of plant tissues comprising the developing microspores may have an impact on both the rate of maturation of microspores and the physiological competence of these cells to respond to culture. In cereals it has been widely shown that pre-culture of spikes containing the anthers at low temperatures (4° C.-10° C.) for prolonged periods of up to 28 days is needed for optimal response. In contrast, studies with dicotyledonous species show cold pre-culture is either ineffective or inhibitory. However, dicotyledonous species such as Brassica may be favourably influenced by temperate or cool growth conditions of donor plants.

Saponaria plants were grown under two initial temperature regimes of 20/15° C. and 10/5° C. with a 16 hour photoperiod to test for donor plant effects. In contrast to Brassica, plants grown at the lower or moderate temperatures were shown to be less or entirely unresponsive.

The composition of the basal medium is another factor that has been shown to influence the response of isolated microspores to culture. In order to optimize the culture medium for Saponaria, a number of different basal medium compositions were evaluated that included: NLN, NN, B5, MS, and K & M (Lichter, R., Z. Pflanzenphysiol. 105:427-434, 1982; Nitsch, J. P., et al., Science 163:85-87, 1969; Gamborg, O. L., et al., Expt. Cell. Res. 50:151-159, 1968; Murashige, T., et al., Physiol. Plant. 15:473-497, 1962; Kao, K. N., et al., Planta. 126:105-110, 1975).

In contrast to species such as tobacco where microspore response is stimulated by less than optimal nitrogen content in the medium (Kyo, M., et al., Plant Physiol. 79:90-94, 1985), best results for Saponaria microspores were achieved with full strength NLN-13 medium. As discussed below, examples of other suitable media will be well known to one of skill in the art. Exemplary suitable media or media with a composition that allows for development of microspore-derived embryos include but are by no means limited to NN-13 and half-strength NLN-13.

One of the key elements of medium composition that has been shown to influence microspore embryo formation is the type and concentration of sugars. Cereals in particular have been shown to benefit from inclusion of sugar alcohols such as mannitol or disaccharides such as maltose whereas dicotyledonous species typically respond best to sucrose. The concentration of sugars in the medium has also been shown to have pronounced effects on the response of cultured isolated microspores. The highest frequency of embryogenesis was observed with melezitose or sucrose, with an osmolarity similar to 10% to 15% sucrose. As discussed below, a wide variety of sugar or sugar sources may be used to supplement the media. Preferably, the sugar or sugar source is not a monosaccharide but is a multi-saccharide, for example, a disaccharide or a trisaccharide. Thus, the media may be supplemented with 10-15% of a higher sugar, for example, a multi-saccharide sugar, for example, a disaccharide or a trisaccharide, for example, sucrose, maltose, melibiose, melezitose or other suitable sugars or sugar sources known in the art or combinations thereof.

A second key element that has been established as pivotal in the induction of embryos from isolated microspores in other species has been the duration and degree of the high temperature treatment needed to reprogram microspore development and induce the formation of embryos. It has been demonstrated that cultured microspores subjected to elevated temperatures undergo a typical heat shock response (Fabijanski S. F., et al., Plant Cell Tissue and Organ Culture 26:203-212, 1991) directly upon exposure to elevated temperatures, protein synthesis activities decline to low levels and the mitotic division of the nucleus is arrested. The duration and degree of temperature treatment for optimal embryo formation varies with individual species and may also vary with individual cultivars. That is, it is to be understood that to achieve the best possible results for one particular genotype, some ‘tweaking’ of the protocol may be necessary in order to obtain optimization for a specific genotype. Generally cereals benefit from incubations in the lower range of 25° C.-28° C. and non-cereals a higher range of 30° C.-35° C. The duration of elevated temperature incubation needed for optimal response, before a return to more ambient temperatures, may vary from 1 to 7 days. For Saponaria the optimal temperature regime for embryo induction is 32° C. for 3 days, although as discussed below, a temperature range of 32° C.+/−2° C. is suitable. Thus, the elevated temperature may be 30-34° C., 30.5-34° C., 31-34° C., 31.5-34° C., 30-33.5° C., 30-33° C., or 30-32.5° C. Furthermore, as discussed below, it is important to note that ‘3 days’ as used herein does not necessarily mean '72 hours, as suitable incubation time periods include but are by no means limited to 60-84 hours, 64-80 hours and 68-76 hours.

Another factor that may influence the ability of microspores to respond to culture is the density of cells per volume of medium. It has been demonstrated for example that the optimal density for B. napes microspores is in the 40,000-100,000 microspores per ml culture medium range (Fan, Z., et al., Protoplasma, 147:191-199, 1988; Kott, L. S., et al., Can. J. Bot. 66:1665-1670, 1988; Polsoni, L., et al., Can. J. Bot. 66:1681-1685, 1988).

Studies were conducted with Saponaria microspores in concentrations ranging from 6,000 to 100,000 cells per ml culture medium. In contrast to results observed obtained with B. napus, the optimal density of cultured microspores for Saponaria was 12,500 to 25,000 cells per ml culture medium.

In addition to sugars and standard inorganic nutrients required for the growth of cells in culture, biochemicals and reagents with known physiological function may also be added to isolated microspore cultures to either encourage embryo formation and development or counteract or sequester inhibitors of embryo growth and development. Examples of substances that have been found beneficial include auxins, cytokinins, gibberellic acid, AgNO₃ or other ethylene antagonists, activated charcoal, and gametocidal chemicals. Additionally, the response of microspores may be enhanced by unknown naturally produced substances from feeder cells or ovules. The range of different potential additives is large and any influence positive or negative must be determined experimentally. For systems that respond poorly, the addition of auxins and or cytokinins is a logical strategy for medium modification.

Isolated microspores of Saponaria were exposed to media enhanced with physiological concentrations of BAP, kinetin, GA3, TDZ, IBA, picloram, cobalt chloride, colchicine, or caffeine. In contrast to other species such as cereals and B. oleracea (Arnison, P. G., et al., Plant Cell, Tissue and Organ Culture 20:217-222, 1990), no stimulation of embryo formation was observed with any of the above substances.

A final factor that has been shown to influence the response of isolated microspores to culture is genotype of the parent material. Although anther and microspore culture has been successful with many species, often the results are achieved only with certain cultivars or genotypes indicating that as yet undefined genetic parameters influence the success of the culture process.

Five genotypes, two commercially available ornamental cultivars and three wild type accessions of Saponaria vaccaria were tested for response to isolated microspore culture. At least some embryos were recovered from all lines tested however the best response was clearly demonstrated by the cultivar “White Beauty” and the accession originating in Finland.

The examples set forth below are for the purposes of illustration and are in no way intended to limit the scope of the invention.

### EXAMPLES

### Example 1

### Determination of Optimal Bud Size for Isolated Microspore Culture of Saponaria vaccaria

Plants of Saponaria vaccaria cv. White Beauty and Pink Beauty, obtained from CN Seeds, Denmark House, Pymoor, Ely, Cambridgeshire, CB6 2EG, UK, three accessions obtained from USDA Regional Plant Introduction Station, Ames, Iowa, PI 304488 (originally from Turkey), PI 578121 (Finland), and Pi 597629 (Mongolia) were grown to maturity in growth chambers with a growth regime of 20/15° C. and a 16 hr photoperiod with a light intensity of 2000 μmol s⁻¹ m⁻¹ (see details in Example 2).

Buds were measured and put into separate Lipshaw baskets according to size (3-3.9, 4-4.9, 5-5.9, 6-6.9, 7-7.9, 8-8.9 mm). The Lipshaw baskets were immersed for one minute in 70% ethanol in a sterile beaker and placed on a shaker. After one minute, the ethanol was removed and the baskets containing the buds were rinsed with sterile water. The Lipshaw baskets were then immersed for 15 minutes in 6% sodium hypochlorite in a sterile beaker placed on a shaker. After 15 minutes, the sodium hypochlorite was removed by three 5-minute washes with sterile water. The buds were removed from the Lipshaw baskets with sterile forceps and placed in a mortar with 5 ml of half strength Gamborg B5 medium with 13% sucrose. The buds were gently crushed with a pestle, the resulting suspension was filtered through a 44 μm nylon screen cloth into a 50 ml sterile centrifuge tube. The mortar and filter were rinsed three times with 5 ml of half strength B5-13 which was filtered and added to the suspension for a total of 20 ml. The suspension was centrifuged at 130-150 g for three minutes. The resulting supernatant was removed and 5 ml of half strength B5-13 was added to the pellet. This procedure was repeated two additional times.

Isolated microspores were cultured at 32° C. for three days.

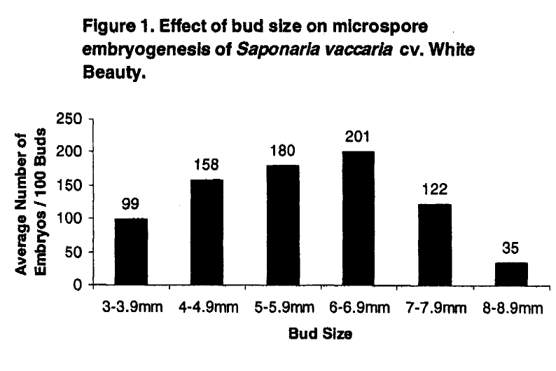

Results achieved that demonstrate the optimal size of flower buds' for isolated microspore culture is 4-7.9 mm are shown in FIG. 1. As discussed above, it is important to note that embryos can still be obtained from buds 3.0-3.9 mm and 8-8.9 mm.

Microscopic examinations of the development of microspores from the different sized buds also suggest that microspore development is quite variable and that some microspores at the appropriate stage for induction are present in a broad range of bud sizes.

### Example 2

### Influence of Donor Plant Growth Temperature on Microspore Culture Response

Microspore donor plants of Saponaria cultivars White Beauty and Pink Beauty were grown as follows. Seeds were planted in 15 cm plant pots filled with commercial greenhouse soil-less mix (e.g. Redi-Earth soil-mix). Pots were placed in growth cabinets with a 16-hour photoperiod and a day/night temperature of 20/15C and a light intensity of 2000 μmol s⁻¹ m⁻¹. Plants were fertilized with 14-14-14 Nutricote 100 (slow release fertilizer) and watered three times a week with 0.35 g L⁻¹ of 15-15-18 (15% N, 15% P, 18% K). Prior to bolting, half of the plants were transferred to a growth cabinet set at 10/5° C.

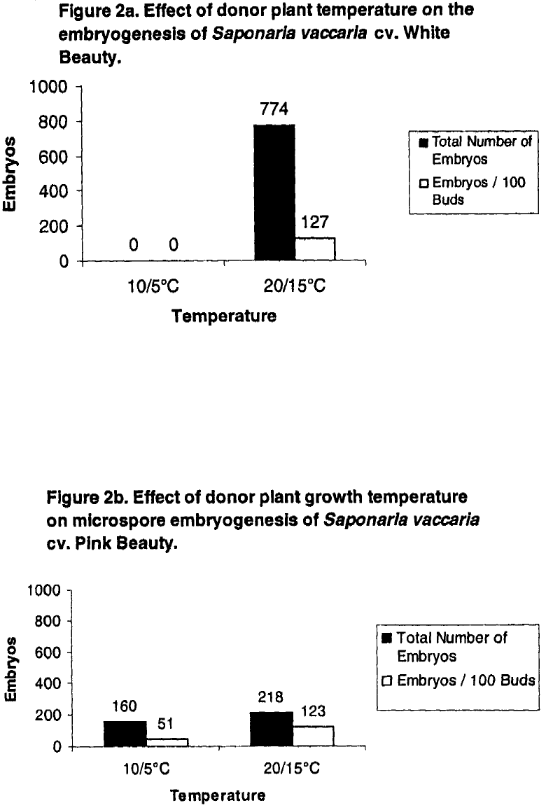

Results shown in FIGS. 2a and 2b demonstrate that cool growth temperatures were either inhibitory or less inductive to embryo formation. For the cv White Beauty, microspore-derived embryos were only obtained from the plants grown at 20/15C. No embryos were produced from the plants grown at the lower temperatures (10/5).

### Example 3

### Influence of Basal Medium Composition on Embryo Formation by Isolated Microspores of Saponaria

Microspores were isolated as described above using Gamborg's B5-13 wash. Five different culture media were tested: B5, MS, NN and ½ strength NLN and full strength NLN (control). 13% sucrose was added to all media. Results presented in Table 1 show full strength NLN medium to be the most stimulatory.

TABLE 1
Effect of basal media on microspore embryogenesis
of Saponaria vaccaria cv. White Beauty

| | Average number |
|---|---|
| | of embryos/100 |
| Media | buds |
| B5-13 | 3 |
| MS-13 | 0 |
| NN-13 | 19 |
| ½ NLN-13 | 60 |
| NLN-13 | 90 |

### Example 4

### Influence of Sugar Composition and Concentration on Embryo Formation by Isolated Microspores of Saponaria vaccaria cv. White Beauty

Six carbohydrates (fructose, glucose, maltose, melibiose, melezitose, and sucrose) at four concentrations equivalent to the osmolarity of 10%, 13%, 15%, and 17% sucrose (+/−20 mOsm) were evaluated for an effect on embryo induction from isolated microspores in NLN culture medium. The different sucrose concentrations were made first and the osmolarity was determined using an Advanced Instruments, Micro Osmometer (model 3300). The 20 different carbohydrate-containing media were then formulated with each having osmolarity equivalent to the four concentrations of sucrose (+/−20 mOsm).

TABLE 2
Effect of different carbohydrates and concentration
on microspore embryogenesis of Saponaria vaccaria

| | | | Ave. No. |
|---|---|---|---|
| Type and percent of | Osmolarity | Actual % | Embryos/100 |
| Carbohydrate | (mOsm) | CHO | Buds |
| Fructose | 349 | 6.2 | 3 |
| Fructose | 472 | 8.4 | 1 |
| Fructose | 545 | 9.8 | 3 |
| Fructose | 642 | 10.8 | 70 |
| Glucose | 337 | 5.4 | 0 |
| Glucose | 476 | 7.6 | 6 |
| Glucose | 544 | 8.8 | 5 |
| Glucose | 624 | 10 | 19 |
| Maltose | 369 | 11 | 26 |
| Maltose | 482 | 15 | 47 |
| Maltose | 566 | 17 | 65 |
| Maltose | 645 | 18.4 | 82 |
| Melibiose | 347 | 11 | 53 |
| Melibiose | 455 | 13 | 100 |
| Melibiose | 550 | 16 | 30 |
| Melibiose | 619 | 18.3 | 33 |
| Melezitose | 355 | 14.5 | 130 |
| Melezitose | 460 | 21.5 | 130 |
| Melezitose | 560 | 26 | 117 |
| Melezitose | 609 | 30.5 | 119 |
| Sucrose | 342 | 10 | 102 |
| Sucrose | 472 | 13 | 101 |
| Sucrose | 549 | 15 | 186 |
| Sucrose | 622 | 17 | 91 |

At least a small number of embryos were observed for each of the carbohydrates used and at each of the concentrations except the lowest concentration of glucose (5.4% glucose which had an osmolarity equal to 10% sucrose).

The results indicate that the monosaccharides (i.e. fructose, glucose), at the concentrations tested, were only weakly beneficial for microspore embryogenesis of Saponaria. Embryos were produced, but frequency of embryogenesis was low compared to the other carbohydrates evaluated. Embryogenesis was higher in media comprising maltose and melibiose (disaccharides). The highest frequency of embryogenesis was observed in media comprising melezitose (trisaccharide) and sucrose (disaccharide). For further medium refinement studies sucrose was used as the source of carbohydrate.

### Example 5

### Effect of Duration of Elevated Temperature Treatment

Microspore cultures were prepared as described in Example 1 and incubated at four different culture temperatures (24, 30, 32, 35° C.). Plates with microspores incubated at 30, 32, or 35° C. were transferred to 24° C. after three days. Additional experiments were conducted in which the duration of incubation at 32° C. was 1, 2, 3, 4, 7, 14 days whereafter plates were maintained at 24° C. for the remainder of three weeks. The plates at 24° C. were maintained for 21 days at 24° C. The results presented in Table 3 below show that the optimal temperature regime for embryo induction is 32° C. for three days.

TABLE 3

| | | |
|---|---|---|
| | | Average number |
| | Total Number | of Embryos/100 |
| Culture Conditions | of Embryos | Buds |

Effect of culture conditions on microspore
embryogenesis of Saponaria vaccaria cv. White Beauty

| | | |
|---|---|---|
| 24° C. - Continuous | 0 | 0 |
| 30° C. - 3 days | 0 | 0 |
| 32° C. - 3 days | 829 | 99 |
| 35° C. - 3 days | 0 | 0 |
| 1 day @ 32° C. | 0 | 0 |
| 2 day @ 32° C. | 0 | 0 |
| 3 day @ 32° C. | 828 | 120 |
| 4 day @ 32° C. | 0 | 0 |
| 7 day @ 32° C. | 1 | 1 |
| 14 day @ 32° C. | 0 | 0 |

Effect of culture conditions on microspore
embryogenesis of Saponaria vaccaria cv. Pink Beauty

| | | |
|---|---|---|
| 24° C. - Continuous | 0 | 0 |
| 30° C. - 3 days | 0 | 0 |
| 32° C. - 3 days | 450 | 36 |
| 35° C. - 3 days | 0 | 0 |
| 1 day @ 32° C. | 0 | 0 |
| 2 day @ 32° C. | 0 | 0 |
| 3 day @ 32° C. | 392 | 52 |
| 4 day @ 32° C. | 0 | 0 |
| 7 day @ 32° C. | 56 | 10 |
| 14 day @ 32° C. | 2 | 2 |

### Temperature Variation Here

As will be appreciated by one of skill in the art, for example ‘3 days’ does not necessarily mean 72 hours but may mean for example 68-76 hours.

### Example 6

### Optimal Density of Saponaria Microspores for Embryo Generation

Isolated microspores of cv. White Beauty and Pink Beauty were prepared as described in Example 1. Densities were determined using a hemocytometer (Sigma Plant Cell Culture, 1989). White Beauty and Pink Beauty microspores were cultured in NLN-13 medium at five different microspore densities (6250, 12,500, 25,000, 50,000, 100,000 microspores/ml). These plates were then incubated at 32° C. for 3 days.

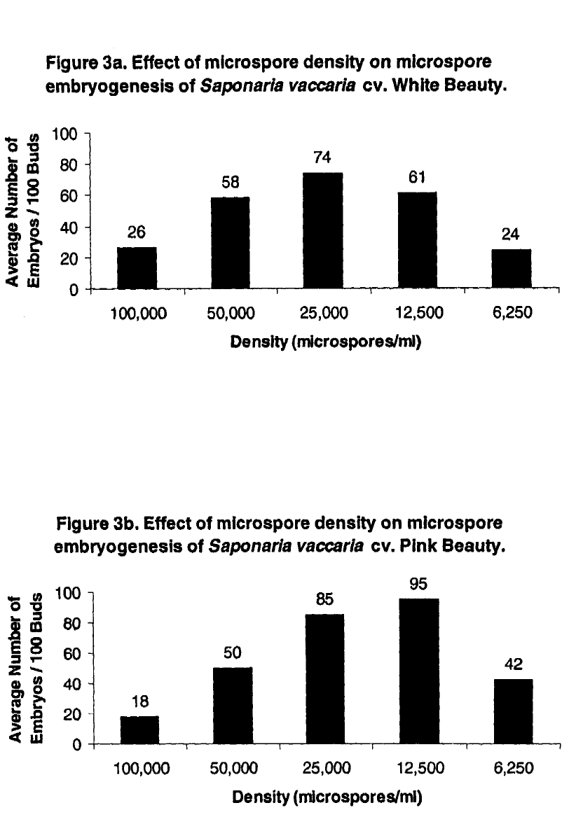

Results presented in FIGS. 3a and 3b show that the preferred density of microspores for optimal induction was in the lower density ranges studied, 12,500 to 25,000, but at least some embryos were recovered at all densities tested.

### Example 7

### Effect of Genotype on Rate of Embryo Formation from Isolated Microspores

Six genotypes were evaluated for response to isolated microspore culture using an inductive regime of 3′ days elevated temperature treatment of 32° C. followed by continuous culture in the dark at 24° C. Embryo formation was scored after 21 days of incubation. The genotypes tested include two commercial ornamental cultivars: White Beauty and Pink Beauty obtained from CN Seeds, Denmark House, Pymoor, Ely, Cambridgeshire, UK. Additionally, accessions designated PI 607444 originating from Israel, PI 578121 originating from Finland, PI597629 originating from Mongolia, and PI 304488 originating from Turkey, were obtained from the USDA North Central Regional Plant Introduction Station.

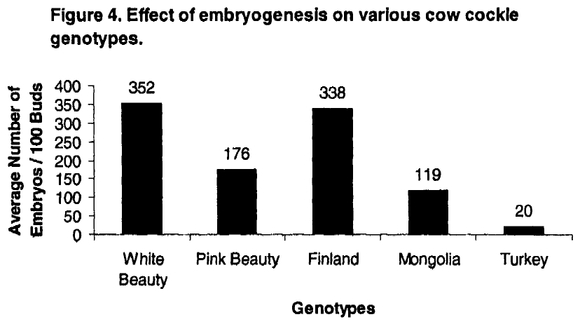

Results presented in FIG. 4 show that embryos were recovered from all lines tested and that the greatest response was observed with cultivar White Beauty and the accession from Finland. The accession that originated from Israel failed to flower and was not included in the FIG. 4. However, it is important to note that if this plant had flowered, there is no reason to believe the protocol would not have worked.

### Example 8

### Demonstration of Double-haploid Lines with Divergent Seed Saponin Profiles

Microspore-derived embryos of Saponaria were plated (10 per dish) on solidified B5 media (Gamborg), free of growth regulators with 1% sucrose and 1% agar. Petri plates were placed in a tissue culture room at 22° C., 16-hour photoperiod and a light intensity of 150 μmol m⁻² s⁻¹. After four weeks, plantlets with well developed root and shoot systems were transplanted directly to soil, all other plantlets were sub-cultured to larger Petri plates for further development (B5 media, no growth regulators, 2% sucrose, 0.8% agar). Once well developed shoot and root systems were formed, the plantlets were transferred to a soil-less mix and grown in the greenhouse or growth cabinet. Plantlets were initially covered with plastic cups to maintain a high humidity. This covering was slowly removed as the plants harden. Optimum growing conditions were used as described in Example 2.

Plantlets were grown to maturity in growth chambers and were found to comprise an approximately equal mixture of haploid and spontaneously doubled-haploid plants. Seed was harvested from doubled haploid lines for compositional analysis.

For analysis, seed was finely ground in a small mortar and a 50 mg portion was placed in a small test-tube with 1.5 ml of 70% methanol containing 15 tag of digitoxin as internal standard. The mixture was stirred occasionally using a vortex shaker or an ultrasonic bath over a period of four or more hours. Solids were removed by centrifugation and the supernatant filtered through a 0.45 μm nylon filter into a sample vial. The sample was chromatographed on a reverse phase column employing a gradient of water/acetonitrile containing 0.12% acetic acid. The gradient was adjusted so that sugars, phenolics, and cyclic peptides eluted prior to saponins which eluted as a class, but which did not overlap the internal standard, digitoxin.

A Water's 2695 Alliance chromatography system with inline degasser, coupled to a ZQ mass detector and a 2995 diode array detector was used for analyses. MassLynx software was used for data acquisition and manipulation. Columns used were a Waters Symmetry RP C18 2.1×150 mm, Sunfire RP C18 2.1×150 mm or equivalent.

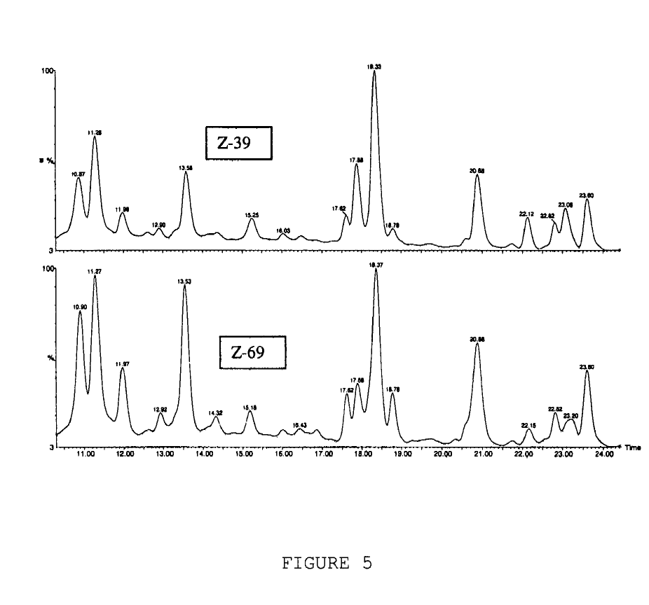

Results presented in FIG. 5 show the comparison of saponin profiles in seed extracts of two doubled haploid lines derived from the ‘Pink Beauty’ cultivar.

For production of a herbal product, a defined and reproducible chemical profile is necessary—otherwise therapeutic benefits would necessarily be variable. Also, some components may be more desirable while others may be undesirable. A true-breeding line having a high titre of desirable component(s) and low titre of undesirable component(s) would be commercially advantageous. The profiles in FIG. 5 illustrate that lines derived from the same species can have different profiles. One can make use of these differences by screening true breeding lines for desirable profiles and make use of this germplasm to develop elite plants.

Hplc chromatograms (saponin region) of seed extracts show significant compositional differences. Quantification of the differences and identity of saponins is presented in detail below in Table 4.

TABLE 4
Relative Amounts of Major Saponin Components in Seed
Extracts of Double Haploid lines Z-39 and Z-69.

| | | | Z-39 | Z-69 | |
|---|---|---|---|---|---|
| Major Peak | Z-39 | Z-69 | Rel. mol | Rel. mol | |
| No. | Rₜ | Rₜ | % | % | Assignment |
| 1 | 10.88 | 10.90 | 8.7 | 10.5 | Vaccaroside B |
| 2 | 11.28 | 11.28 | 13.7 | 14.3 | |
| 3 | 11.98 | 11.97 | 2.8 | 5.2 | |
| 4 | 13.58 | 13.55 | 8.2 | 11.8 | Vaccaroside E |
| 5 | 17.62 | 17.62 | 2.6 | 2.9 | |
| 6 | 17.88 | 17.90 | 9.3 | 4.3 | |
| 7 | 18.33 | 18.37 | 21.3 | 15.4 | Segetoside I |
| 8 | 20.88 | 20.88 | 9.0 | 9.1 | Segetoside I Ac |
| 9 | 22.12 | 22.15 | 3.1 | 1.4 | Segetoside H |
| 10 | 23.60 | 23.60 | 5.7 | 6.3 | |

Relative amounts determined from HPLC chromatogram with photodiode array detection at 205-210 nm. Identification of known saponins was based on extracted mass spectrum.

### Example 9

### Demonstration of Double-haploid Lines with Divergent Seed Cyclopeptide Profiles

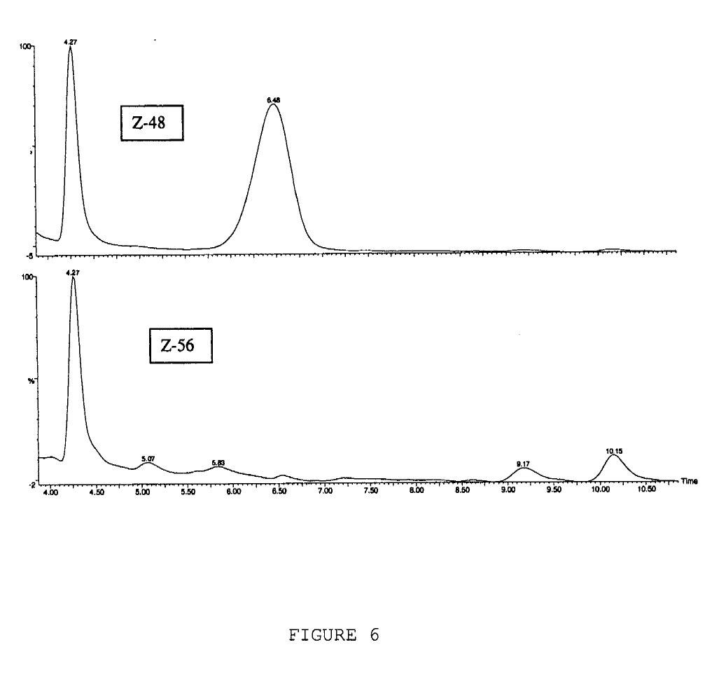

Microspore-derived doubled haploid plants were produced and seed grown and prepared for analysis as described in Example 8. Doubled-haploid lines with differing composition of indole containing cyclopeptides were discovered as shown in FIG. 6 and below in Table 5.

Biological activity is directly related to a compound's chemical structure and it is known that even small structural changes can result in significant augmentation or attenuation of activity. The ability to produce true breeding lines from which can consistently be obtained desired chemicals at desired levels, or reproducible mixtures of chemicals, confers a significant benefit to production of these products. Conversely, it is known that extracts from plants may contain components possessing untowards effects, and it would thus be desirable to have true-breeding plants which produce reduced levels of these components. Also, for compounds to be useful as starting materials or precursors for semi-synthetic preparation of medicinals, it would be desirable to have high levels of that component produced without contamination of closely related materials which might make isolation and purification difficult. For example in Table 5 line Z-39 would be superior to line Z-56 for production of cyclic peptide segetalin A.

TABLE 5
Relative Amounts of Major Tryptophan Containing
Cyclopeptides, Segetalins A and B, in Seed Extracts
of Double Haploid Lines Z-48 and Z-56.

| | | | Z-39 | Z-69 | |
|---|---|---|---|---|---|
| Major Peak | Z-48 | Z-56 | Rel. mol | Rel. mol | |
| No. | Rₜ | Rₜ | % | % | Assignment |
| 1 | 4.27 | 4.27 | 31.9 | 68.1 | Segetalin B |
| 2 | 6.48 | 6.53 | 97.2 | 2.8 | Segetalin A |

### REFERENCES

### Each of Which is Incorporated in its Entirety by this Reference

Amison, P. G., et al., Genotype specific response of cultured broccoli (B. oleracea var italica) anthers to cytokinins. Plant Cell, Tissue and Organ Culture 20:217-222 (1990).
Barro, F., et al., Response of different genotypes of Brassica carinata to microspore culture. Plant Breeding 118:79-81 (1999).
Biliaderis, C. G., et al., Composition and physio-chemical properties of starch from cow cockle (Saponaria vaccaria L.) seeds. Starch/Stärke 45:121-127 (1993).
Cordewener, J. H. G. et al., Induction of microspore embryogenesis in Brassica napus L. is accompanied by specific changes in protein synthesis. Planta. 195:50-56 (1994).
Dunwell, J. M. Pollen, ovule and embryo culture as tools in plant breeding. In: Plant Tissue Culture and its Agricultural Applications, L. A. Withers and P. G. Alderson (eds) Butterworth, London (1986).
Fabijanski, S. F., et al., Heat shock response during anther culture of broccoli (B. oleracea var italica). Plant Cell, Tissue and Organ Culture 26:203-212 (1991).
Fan, Z., et al., Development of microspores in vivo and in vitro in Brassica napus L. Protoplasma 147:191-199 (1998).
Ferrie, A. M. R., et al., Biotechnology applications for haploids. In: Biotechnological Applications of Plant Cultures. Shargool, P. D. and Ngo, T. T. (eds.), CRC Press, Boca Raton, p 77-110 (1994).
Ferrie, A. M. R., et al., Evaluation of Brassica rapa L. genotypes for microspore culture response and identification of a highly embryogenic line. Plant Cell Reports 14:580-584 (1995).
Ferrie, A. M. R., et al., Microspore culture for haploid plant production. In: O. L. Gamborg and G. G. Phillips (eds.) Plant Cell, Tissue and Organ Culture: fundamental method. Springer: Berlin, pp. 155-164 (1995).
Ferrie, A. M. R., et al., Brassica improvement through microspore culture. In: Biotechnology in Agriculture and Forestry Vol. 54: Brassica. E. C. Pua and C. J. Douglas. Berlin, Springer-Verlag, 149-168 (2004).
Ferrie, A. M. R., et al., Microspore embryogenesis of high sn-2 erucic acid Brassica oleracea germplasm. Plant Cell, Tissue and Organ Culture 57:79-84 (1999).
Ferrie, A. M. R., et al., Haploid Embryogenesis. In: In Vitro Embryogenesis in Plants, Thorpe, T. A. ed. Kluwer Academic Publishers, Dordrecht, pp. 309-344 (1995).
Gamborg, O. L., et al., Nutrient requirements of suspension cultures of soybean root cells. Exp. Cell Res. 50:151-158 (1968).
Gaillard, A., et al., Optimization of maize microspore isolation and culture conditions for reliable plant regeneration. Plant Cell Reports 10:55-58 (1991).
Kao, K. N., et al., Nutritional requirements for growth of Vicia hajastana cells and protoplasts at a very low population density in liquid media. Planta. 126:105-110 (1975).
Keller, W. A., et al., High frequency production of microspore-derived plants from Brassica napes anther cultures. Z. Pflanzenzuchtg. 80:100-108 (1978).
Kott, L. S., et al., Cytological aspects of isolated microspore culture of Brassica napus. Can. J. Bot. 66:1658-1664 (1988).
Kyo, M. et al., Studies on conditions for cell division and embryogenesis in isolated pollen culture in Nicotiana rustica. Plant Physiol. 79:90-94 (1985).
Lionneton, E., et al., Improved microspore culture and doubled-haploid plant regeneration in the brown condiment mustard (Brassica juncea). Plant Cell Reports 20:126-130 (2001).
Lichter, R. Induction of haploid plants from isolated pollen of Brassica napus. Z. Pflanzenphysiol. 105:427-434 (1982).
Maheshwari, S. C., et al., Haploids from pollen grains—retrospect and prospect. Amer. T. Bot. 69:865-879 (1982).
Morita, H., et al., Segetalins B, C, D; three new cyclic peptides from Vaccaria segetalis. Tetrahedron 51:6003-6014 (1994).
Morita, H., et al., Conformational analysis of hexapeptide, segetalin A from Vaccaria segetalis. Tetrahedron 51:5987-6002 (1995).
Murashige, T. et al., A revised medium for rapid growth and bioassays with tobacco tissue cultures. Physiol. Plant. 15:473-497 (1962).
Mazza, G., et al., Compositional and morphological characteristics of cow cockle (Saponaria vaccaria) seed, a potential alternative crop. J. Agric. Food Chem. 40:1520-1523 (1992).
Nitsch, J. P., et al., Haploid plants from pollen grains. Science 163:85-87 (1969).
Polsoni, L., et al., Large-scale microspore culture technique for mutation-selection studies in Brassica napus. Canadian Journal of Botany 66:1681-1685 (1988).

| | | |
|---|---|---|
| U.S. Pat. No. 4,438,148 | March 1984 | O'Rourke et al. |
| U.S. Pat. No. 4,684,479 | August 1987 | D'Arryo |
| U.S. Pat. No. 4,840,906 | June 1989 | Hunter |
| U.S. Pat. No. 4,911,946 | March 1990 | Singer et al. |
| U.S. Pat. No. 5,153,020 | October 1992 | Singer et al. |
| U.S. Pat. No. 5,322,789 | June 1994 | Genovesi et al. |
| U.S. Pat. No. 5,370,894 | December 1994 | Singer |
| U.S. Pat. No. 5,445,961 | August 1995 | Genovesi et al. |
| U.S. Pat. No. 5,453,281 | Sepember 1995 | Whistler |
| U.S. Pat. No. 5,492,827 | February 1996 | Dirks |
| U.S. Pat. No. 5,503,766 | April 1996 | Kulperger |
| U.S. Pat. No. 5,639,951 | June 1997 | Bosemark et al. |
| U.S. Pat. No. 5,662,937 | September 1997 | McCuaig |
| U.S. Pat. No. 5,900,375 | May 1999 | Simmonds et al. |
| U.S. Pat. No. 6,051,154 | April 2000 | Meyer |
| U.S. Pat. No. 6,200,808 | March 2001 | Simmonds et al. |
| U.S. Pat. No. 6,267,970 | July 2001 | Matesevac et al. |
| U.S. Pat. No. 6,362,393 | March 2002 | Konzak et al. |
| U.S. Pat. No. 6,482,770 | November 2002 | Dutcheshen et al. |
| U.S. Pat. No. 6,485,775 | November 2002 | Camin et al. |
| U.S. Pat. No. 6,743,752 | June 2004 | Dutcheshen |
| U.S. Pat. No. 6,764,854 | July 2004 | Konzak et al. |
| U.S. Pat. No. 6,812,028 | November 2004 | Kasha et al. |

1. A method of producing a microspore-derived doubled haploid plant of Saponaria vaccaria comprising:
growing microspore donor plants under conditions that allow development of microspores competent for development into embryos:
isolating the microspores from flower buds comprising the micropores at a developmental stage competent for inducing embryo development;
culturing the isolated microspores in a medium with a composition that allows for development of microspore-derived embryos;
exposing the cultured isolated micropores to an elevated temperature for inducing embrogenesis;
recovering embryos; and
generating doubled haploid plants.

2. The method according to claim 1, wherein the embryos are isolated from buds having a bud size of 3-9 mm.

3.-7. (canceled)

8. The method according to claim 1, wherein the embryos are isolated from buds having a bud size of 4-7.9 mm.

9. The method according to claim 1, wherein the elevated temperature is between 30-34° C.

10. The method according to claim 1, wherein the medium includes a non-monosaccharide sugar at 10% (v/v) or greater.

11. The method according to claim 1, wherein the medium includes a non-monosaccharide sugar at 10-30.5% (v/v).

12. The method according to claim 1, wherein the medium includes a non-monosaccharide sugar at 10-15% (v/v).

13. The method according to claim 1, wherein the donor plant is grown in a growth regime of 20/15° C. day/night.

14. The method according to claim 1, wherein the culturing is done for 3 days at a temperature in a range of from 30-34° C.

15. The method according to claim 14, wherein the temperature is 32° C.

16. The method according to claim 1, wherein the generated doubled haploid Saponaria vaccaria plant contains an altered chemical composition compared to a parental Saponaria vaccaria plant.

17. The method according to claim 1, wherein the generated doubled haploid Saponaria vaccaria plant contains increased content of saponin, starch or both saponin and starch compared to a parental Saponaria vaccaria plant.

18. A doubled haploid Saponaria vaccaria plant made according to the method of claim 1.

19. The doubled haploid plant according to claim 18, containing increased content of saponin, starch or both saponin and starch compared to a parental Saponaria vaccaria plant.
